(19) United States
(12) Patent Application Publication
(10) Pub. No.: US 2006/0106317 A1
(43) Pub. Date: May 18, 2006

(54) Optical system and use thereof for detecting patterns in biological tissue

(75) Inventors: Peter R.H. McConnell, Vancouver, BC (CA); Bruce W. Adams, North Vancouver (CA)

Correspondence Address: GOTTLIEB RACKMAN & REISMAN PC, 270 MADISON AVENUE, 8TH FLOOR, NEW YORK, NY, 100160601, US

(73) Assignee: Joule Microsystems Canada Inc., Delta, BC (CA)

(21) Appl. No.: 10/527,857

(22) PCT Filed: Sep. 16, 2003
(86) PCT No.: PCT/CA03/01372

§ 371 Date: Nov. 2, 2005

(30) Foreign Application Priority Data
Sep. 16, 2002 (CA) 2,403,748

(51) Int. Cl. A61B 6/00

(52) U.S. Cl. 600/476

(57) ABSTRACT

The present invention provides an optical system comprises a scanning spectrometer incorporating an electronic light modulator and digital signal processing means. The spectrometer technique combined with optical signal encoding provides the ability to obtain spectral signatures and identify optical patterns of biological tissue. One example of biological tissue scanning includes techniques such as identification of optical patterns of normal and abnormal tissues in addition to the delineation of these spectral patterns between the abnormal and normal tissue. Due to an enhanced signal-to-noise ratio provided by the optical system according to the present invention, this optical system can detect patterns in biological tissue that may be more subtle than those patterns that would be possible to obtain with currently available optical systems.

### FIELD OF THE INVENTION

This present invention pertains to the field of optical systems, and in particular to optical systems used for detecting patterns in biological tissue.

### BACKGROUND OF THE INVENTION

Currently, clinical diagnosis of skin disease is generally accomplished by visual inspection under white light illumination. In this process, the reflectance light of a skin lesion is examined. Visual diagnosis alone may not be particularly accurate for early detection of skin cancer since many skin conditions have a similar appearance under white light. Therefore, when a suspect lesion is identified by visual examination, a biopsy is often performed for a definitive diagnosis. This is because it is crucial to diagnose skin pre-cancer or cancer at an early stage when it is curable. Thus, it is important to improve the clinical diagnosis of suspected skin lesions so as to avoid unnecessary skin biopsies.

Several approaches have been tried to improve dermatological diagnosis. Digital processing of reflectance images has been extensively investigated recently. Although reflectance imaging has led to improvements in the registration, recording, and documentation of skin lesions, there has been little improvement in the diagnostic accuracy. The foregoing approach does not provide any additional data to the physician making the visual assessment because it is still based on the reflectance pattern of a lesion under white light illumination, which is essentially the same pattern a human observer sees.

An alternative approach is ultraviolet (UV) or infrared (IR) photography that does extend the visual perception of a physician to the UV or IR reflectance patterns. However, the inconvenience due to delays in processing of film images containing these reflectance patterns, renders this technique impractical for everyday use.

A further alternative approach that is already in widespread medical use involves a “Wood's lamp”, which consists of a mercury discharge lamp associated with a filter that transmits UVA light with a 365 nanometer peak while absorbing visible light. When this device is used to assist in skin diagnosis, the eye serves as both the detector and the long pass filter. The eye is not sensitive to UV light, but is sensitive to visible fluorescence light when the “Wood's lamp” is used in a darkened room, where the physician can see an image of a fluorescing disease site, for example. The “Wood's lamp” is useful for the diagnosis of some skin conditions such as tinea capitis, tinea versicolor, erythrasma, and some pseudomonas infections, as well as aiding in the detection and diagnosis of hypopigmented skin. It is of no value in conditions where the emitted fluorescence is not in the visible spectrum because the human eye cannot detect such fluorescence. It is also incapable of detecting Raman scattering. Thus, there has gone unmet a need for apparatus and methods that are able to detect and analyze fluorescence both within and beyond the visible spectrum, and that can use fluorescence, reflectance and/or Raman scattering to identify, and distinguish between, a variety of skin diseases.

There are a number of spectrophotometers for use in medical diagnosis. For example, U.S. Pat. No. 6,069,689 describes an apparatus for diagnosis of a skin disease site using spectral analysis includes a light source for generating light to illuminate the disease site and a probe unit optically connected to the light source for exposing the disease site to light to generate fluorescence and reflectance light. The probe unit also collects the generated fluorescence and reflectance light and transmits this light to a spectrometer to be analyzed. The spectrometer generates and displays spectral measurements of the fluorescence light and the reflectance light, which together assist the user in diagnosing the disease site. The apparatus makes use of a conventional personal computer using a plug-in spectrometer card to provide a compact and low cost system. The system performs combined fluorescence and reflectance spectral analysis in a quick and efficient manner to provide a tool for dermatological diagnosis. This device however can have limited detection capabilities due to noise entering the system, in terms of both stray light parameters and internal electronic noises. Depending on the noise level the fluorescence that is being produced by the skin tissue may not be identifiable over the noise within the system.

U.S. Pat. No. 6,055,451 describes an apparatus and method that includes utilizing a device intended to be inserted into a patient's body to determine a characteristic of a target tissue. In one apparatus and method, a device illuminates the target tissue with amplitude modulated excitation electromagnetic radiation, and the device senses returned electromagnetic radiation. A phase shift between the excitation and return electromagnetic radiation is determined, and the phase shift is used to determine characteristics of the target tissue. A demodulation factor, representing ratios of the AC and DC components of the excitation and return electromagnetic radiation may also be calculated and used to determine characteristics of the target tissue. In another apparatus and method embodying the invention, a device illuminates a target tissue with polarized electromagnetic radiation, and a return electromagnetic radiation is sensed. The amplitude of the returned electromagnetic radiation is sensed in mutually perpendicular planes, and this information is used to determine an anisotropy factor. The anisotropy factor, in turn, is used to determine characteristics of the target tissue. In either of the above-described methods, the return radiation could be a portion of the excitation radiation that has been reflected or scattered from the target tissue, or the returned electromagnetic radiation could be fluorescent emissions generated by endogenous or exogenous fluorophores located in the target tissue.

An apparatus and method for imaging diseases in tissue are presented in U.S. Pat. No. 5,590,660. The apparatus employs a light source for producing excitation light to excite the tissue to generate autofluorescence light and for producing illumination light to generate reflected and back scattered light (remittance light) from the tissue. Optical sensors are used to receive the autofluorescence light and the remittance light to collect an autofluorescence light image and a remittance light image. A filter acts to integrate the autofluorescence image over a range of wavelengths in which the autofluorescence intensity for normal tissue is substantially different from the autofluorescence intensity for diseased tissue to establish an integrated autofluorescence image of the tissue. The remittance light image provides a background image to normalize the autofluorescence image to account for image non-uniformity due to changes in distance, angle and illumination intensity. A monitor displays the integrated autofluorescence image and the remittance light image to produce a normalized image in which diseased tissue is distinguishable from normal tissue. The optical sensor can be installed adjacent the end of an endoscope probe inserted into a body cavity. A method for imaging diseased tissue using an integrated fluorescence image and a normalizing remittance image is also disclosed.

Based on the above, the use of optical energy maps is becoming recognised as a desirable and non-invasive method of characterising physiological conditions of tissue.

However, there is a need for a new optical system that has the capability of identifying weak signals, in particular the fluorescence of tissue being illuminated thereby enabling the detection of an optical pattern of tissue.

### SUMMARY OF THE INVENTION

An object of the present invention is to provide an optical system and use thereof for detecting optical patterns in biological tissue. In accordance with one aspect of the present invention, there is provided an optical system for detecting optical characteristics of biological tissue, said optical system comprising: a photonic energy source for emitting electromagnetic radiation, wherein said photonic energy source is controlled by a digital signal processing means; an optical emission processing means for receiving electromagnetic radiation from the photonic energy source and transmitting one or more illumination wavelengths to the biological tissue, wherein the optical emission processing means is controlled by the digital processing means; an optics assembly providing a means for aligning emitter optics of the optical emission processing means with detector optics of a received light optical processing means; a received light optical processing means for collecting and isolating one or more wavelengths of received electromagnetic radiation from the biological tissue and transmitting the isolated one or more wavelengths of received electromagnetic radiation to an optical detector, wherein said received light optical processing means is controlled by the digital signal processing means; an optical detector for sensing and converting the isolated one or more wavelengths of received electromagnetic radiation into an electrical signal; and digital signal processing means to perform match filtering of the electrical signal received from the optical detector and for controlling the functionality of the photonic energy source, the optical emission processing means and the received light optical processing means.

In accordance with another aspect of the present invention, there is provided a use of an optical system for generating a pattern of optical characteristics of biological tissue, said optical characteristics being reflectance and fluorescence characteristics of the illuminated biological tissue, said optical system comprising: a photonic energy source for emitting electromagnetic radiation, wherein said photonic energy source is controlled by a digital signal processing means; an optical emission processing means for receiving electromagnetic radiation from the photonic energy source and transmitting one or more illumination wavelengths to the biological tissue, wherein the optical emission processing means is controlled by the digital processing means; an optics assembly providing a means for aligning emitter optics of the optical emission processing means with detector optics of a received light optical processing means; a received light optical processing means for collecting and isolating one or more wavelengths of received electromagnetic radiation from the biological tissue and transmitting the isolated one or more wavelengths of received electromagnetic radiation to an optical detector, wherein said received light optical processing means is controlled by the digital signal processing means; an optical detector for sensing and converting the isolated one or more wavelengths of received electromagnetic radiation into an electrical signal; and digital signal processing means to perform match filtering of the electrical signal received from the optical detector and for controlling the functionality of the photonic energy source, the optical emission processing means and the received light optical processing means.

In accordance with another aspect of the present invention, there is provided a A method for generating a pattern of optical characteristics of biological tissue, said method comprising the steps of: illuminating the biological tissue with one or more encoded and predetermined wavelengths of electromagnetic radiation, in order to generate encoded reflectance and fluorescence from the biological tissue; collecting said generated encoded reflectance and fluorescence associated with the one or more encoded predetermined wavelengths of electromagnetic radiation; decoding said generated encoded reflectance and fluorescence associated with the predetermined one or more wavelengths; repeating steps a) through c) for a next predetermined one or more wavelengths of electromagnetic radiation; generating a pattern of optical characteristics, said pattern being a representation of the reflectance and fluorescence intensities associated with each of the predetermined wavelengths of electromagnetic radiation.

### BRIEF DESCRIPTION OF THE FIGURES

FIG. 1 is a schematic diagram of the optical system components corresponding to one embodiment of the present invention.

FIG. 2 is a schematic diagram of the optical system according to another embodiment of the present invention.

FIG. 3 is a schematic diagram of a optical system according to a further embodiment of the present invention incorporating a Matched Filter Receiver.

FIG. 4 is a schematic diagram of a digital signal processing means and light pulse processing algorithm.

FIG. 5 is a graphical representation of the pattern of difference in reflectance and fluorescence for a metastatic tumour liver tissue vs normal liver tissue from the same individual, wherein the horizontal axis is the detected wavelength and the vertical axis represents the emitted wavelength.

FIG. 6 is a graphical representation of the pattern of difference in reflectance and fluorescence for normal liver tissue from two different individuals, wherein the horizontal axis is the detected wavelength and the vertical axis represents the emitted wavelength.

FIG. 7 is a graphical representation of the pattern of difference in reflectance and fluorescence for non-metastatic basal cell carcinoma skin tissue vs normal skin tissue from the same individual, wherein the horizontal axis is the detected wavelength and the vertical axis represents the emitted wavelength.

FIG. 8 is a graphical representation of the pattern of difference in reflectance and fluorescence for bronchogenic malignant tumour tissue vs normal tissue in close proximity from the same individual, wherein the horizontal axis is the detected wavelength and the vertical axis represents the emitted wavelength.

FIG. 9 is a graphical representation of the pattern of the relative difference in the fluorescence of bronchogenic malignant tumour tissue vs a normal tissue in close proximity at three emission wavelengths.

FIG. 10 demonstrates On-Off keyed signal with a 0 dB signal to noise ratio, using pulse amplitude modulation detection as is used in one embodiment of the present invention.

FIG. 11 demonstrates signal detection using frequency domain detection as is used in one embodiment of the present invention.

FIG. 12 demonstrates the results of the time domain correlation output from binary pulse coding signal detection as is used in one embodiment of the present invention.

FIG. 13 is a schematic representation of a pulse coding channel model according to one embodiment of the present invention.

FIG. 14 depicts the detector output using a linear FM Chirp, which is a 125 msec wide rect function, swept from 500 Hz to 3500 Hz and sampled at 8000 samples/sec according to one embodiment.

FIG. 15 demonstrates the use of a linear FM pulse coding technique where the pulse duration was left at 0.125 seconds and the bandwidth was 1600 Hz for a time bandwidth product (TBP) of 200. A log scale of the detector was calculated as; P=20×log s, where s is the time domain output of the matched filter according to one embodiment.

FIG. 16 demonstrates the use of a linear FM pulse coding technique as in FIG. 15 for a TBP of 800.

FIG. 17 demonstrates the use of a linear FM pulse coding technique as in FIG. 15 for a TBP of 2250.

FIG. 18 is a time domain plot for the case of a TBP of 2250, where the detector amplitude is plotted according to one embodiment.

FIG. 19 is a schematic representation of an optical system according to one embodiment of the present invention.

### DETAILED DESCRIPTION OF THE INVENTION

Definitions

The term, electronic light modulator, means an acousto-optic modulator, mechanical light chopper, hologram, and electrically driven opto-electronics or similar devices.

The term, an illumination light source, means a light emitting diode (LED), incandescent, laser, gas discharge lamp, laser diode, arc lamp, x-ray source or similar devices.

The term, monochromator, means a light-dispersing instrument which is used to obtain light of substantially one wavelength, or at least of a very narrow band of the spectrum and may be for example an interference filter, cutoff filter, diffraction prism, diffraction grating, interferometer, hologram or similar devices.

The term, collecting means, includes diffraction or reflective optics, lenses, mirrors, or optical fibres.

The term, photodetector device, includes photodiode, photomultiplier, charge couple device (CCD).

The phrase, an analog circuit to condition the signal from the photodetector, includes amplifier, DC Level shifter, gain control, and noise prefiltering and like functions.

The term, coding signal, includes amplitude modulated, phase modulated, frequency modulated, and phase and amplitude modulated signal.

The term, resultant radiation, refers to each or all of the reflected, transmitted, absorbed and fluoresced light that result when a subject is exposed to an illuminating radiation.

The phrase, weak signal detection, refers to techniques used to enable measurement of low intensity emission radiation from a sample. For any given signal to noise ratio, increasing the bandwidth used to transfer the information can lower the information error rate. The signal bandwidth is spread prior to transmission in the noisy channel, and then despread upon reception. This process results in what is called Processing Gain.

The term, signal spreading, refers to a number of means of spreading the signal, including Linear Frequency Modulation (sometimes called Chirp Modulation) and Direct Sequence methods.

The term, signal despreading, refers to a process, which is accomplished by correlating the received signal with a similar local reference signal using a Correlation Receiver or Matched Filter receiver technique. When the two signals are matched, the spread signal is collapsed to its original bandwidth before spreading, whereas any unmatched signal is spread by the local reference to essentially the transmission bandwidth. This filter then rejects all but desired signal. Thus, in order to optimize a desired signal within its interference (thermal noise in the detection system, ambient light induced noise, AC line noise, etc.), a matched filter receiver enhances the signal while suppressing the effects of all other inputs, including noise.

The term biological tissue is used to define any type of tissue for example, skin, liver, heart, kidney, lung or any other type of biological tissue as would be readily understood by a worker skilled in the art. Biological tissue further comprises tissue which has normal characteristics or abnormal characteristics for example tissue in a diseased state or abnormal growth state. Biological tissue being evaluated using the present invention can additionally be in situ or a test sample, for example.

Unless defined otherwise, all technical and scientific terms used herein have the same meaning as commonly understood by one of ordinary skill in the art to which this invention belongs.

The optical system comprises a scanning spectrometer incorporating an electronic light modulator and digital signal processing means. The spectrometer technique combined with optical signal encoding provides the ability to obtain spectral signatures and identify optical patterns of biological tissue. One example of biological tissue scanning includes techniques such as identification of optical patterns of normal and abnormal tissues in addition to the delineation of these spectral patterns between the abnormal and normal tissue. Due to an enhanced signal-to-noise ratio provided by the optical system according to the present invention, this optical system can detect patterns in biological tissue that may be more subtle than those patterns that would be possible to obtain with currently available optical systems.

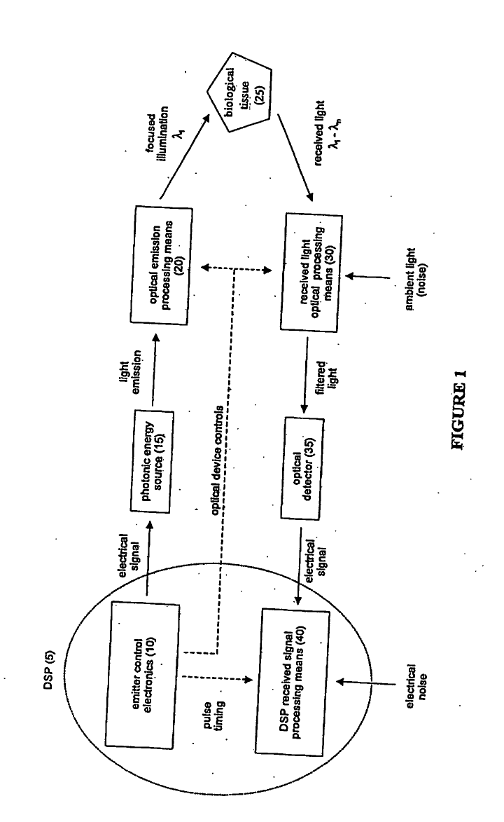

With reference to FIG. 1, the optical system of the present invention comprises a spectrometer and a digital signal processing means 5, comprising: a photonic energy source 15 which is controlled by said digital signal processing means 5 (specifically the emitter control electronics 10), to emit electromagnetic radiation which can range from ultraviolet to far infrared (or bandwidth from 150 to 3000 nm); optical emission processing means 20 which is controlled by said digital signal processing means 5 (specifically the emitter control electronics 10) to receive light from the photonic energy source 15 and to deliver one or more illumination wavelengths in a pulse sequence to biological tissue 25, wherein the optical emission processing means 20 can comprise a means for isolating one or more illumination wavelengths and emitter optics that orient and focus the illumination wavelength(s) onto the biological tissue 25; received light optical processing means 30 which is controlled by said digital signal processing means 5 (specifically the emitter control electronics 10) to collect and isolate one or more wavelengths of received light due to the illumination of the biological tissue 25, wherein the received light optical processing means 30 can comprise detector optics for collecting the received light from the biological tissue 25 and a means for isolating one or more of the wavelengths of the received light; an optical detector 35 to sense and convert to an electrical signal the received light which has been transmitted by the received light optical processing means 30; and a DSP received signal processing means 40, which is a component of the digital signal processing means 5, to perform the match filtering on the output of the optical detector 35, wherein said match filtering is performed based on the received electrical signals from the optical detector 35 and control parameters from the emitter control electronics 10.

There are various locations for noise or interference to enter the system according to the present invention, with this interference decreasing the ability to detect signals received from the biological tissue due to its illumination. For example and with further reference to FIG. 1, ambient light can enter the system through the received light optical processing means 30 and electrical noise can enter the system through the DSP received signal processing means 40. The incorporation of a digital signal processing means can provide a means for the encoding of the illumination signal and the matched filtering of the received signal in relation to the encoded illumination signal, and as such can provide improved detection of the received signals resulting from the illumination of the biological tissue.

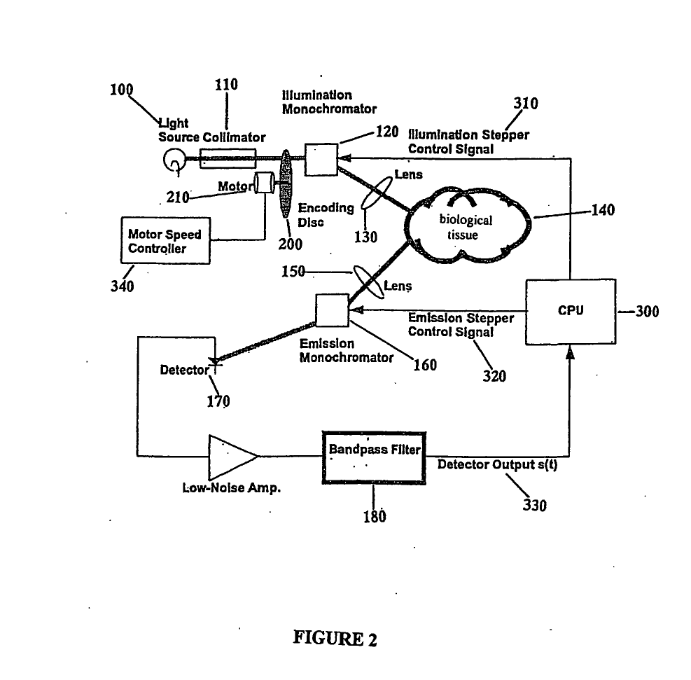

In order to describe how the components operate together, an overview of one embodiment of a system in accordance with this invention is presented in FIG. 2. In this embodiment an illumination light source 100 is controlled by digital signal processing means 300 to emit radiation having a bandwidth ranging from, for example, 250 nm to 1000 nm. A collimator 110 linearises the illumination light and directs it to the light modulator 200, wherein a collimator 110 may be, for example, a long narrow tube in which strongly absorbing or reflecting walls permit only radiation travelling parallel to the tube axis to traverse the entire length. The light modulator 200 which could be an encoding disc (as shown in FIG. 2), acousto-optic modulator, or electronic modulator such that it may enable amplitude or phase modulation, for example, essentially spreading the optical signal. An illumination monochromator 120 is controlled by the digital signal processing means 300 to receive light from the illumination light source 100 and to deliver the N^{th} wavelength in a pulsed sequence to an optical probe which delivers the N^{th} wavelength to the biological tissue 140, for example, biological tissue. The resultant radiation, due to the illumination of the biological tissue, is collected and delivered to the emission monochromator 160. Radiation signals detected from the biological tissue are still encoded with the spread function coding and the intensity is proportional to the reflection coefficient and the fluorescence coefficient. The detection monochromator 160, which is controlled by digital signal processing means 300, separates the reflection and fluorescence spectra optically, by performing specific digital processing tasks to pass the N^{th} wavelength reactive characteristics for a specific illumination wavelength, so that each of these encoded optical signals can be detected by the photo detector 170. The photo detector 170 detects the optical signal and converts it to an electrical signal, which is then processed by the bandpass filter 180, (essentially an Analog to Digital Converter) and transmits it to the digital signal processing means 300. The digital signal processing means 300 performs matched filtering in order to identify and isolate the response of the biological tissue to the illumination radiation from the noise that enters the optical system from various sources.

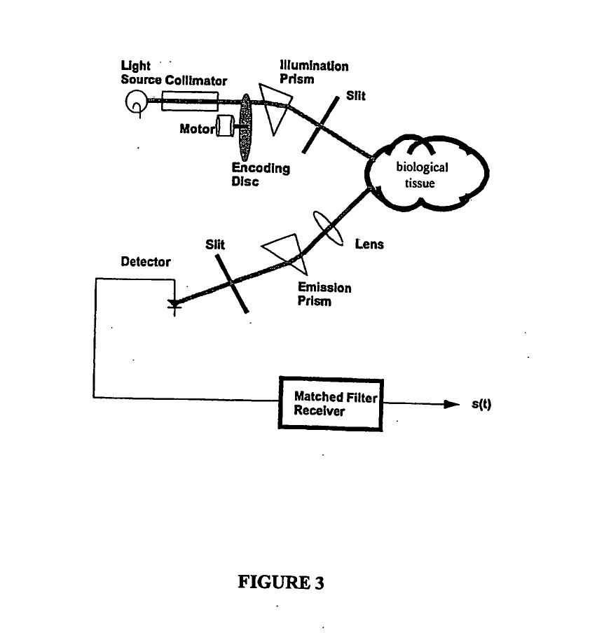

In an alternate embodiment the optical system can be configured with the components as illustrated in FIG. 3. A light source 100 generates photonic energy which is directed towards an encoding disc 200 by a collimator 110. The directed encoded photonic energy passes through an illumination prism 520 for separating the various wavelengths of the illumination radiation. The separated illumination radiation is directed towards a slit 530 oriented in a manner such that the desired wavelength or band of wavelengths are transmitted to the biological tissue 140. The radiation emitted by the biological tissue 140 as a result of its illumination, is collected by a lens 150 and transmitted to an emission prism 540 wherein the emitted radiation is separated into the various wavelengths. The emission prism 540 directs the emitted radiation to a slit 550 oriented in a manner such that the desired wavelength or band of wavelengths is directed towards the detector 170. The detector 170 converts the emission radiation into an electrical signal which is directed to the matched filter receiver 560 for processing the gathered information relating to the illumination of the biological tissue.

There are a number of embodiments of this optical system, comprising different components. Each embodiment, however, has a form of each of these components. Some criteria for choosing which component should be included in a particular embodiment will be described below.

A Photonic Energy Source

Each embodiment includes a photonic energy source, which is controlled by said digital signal processing means to emit electromagnetic radiation, which can range from ultraviolet to far infrared (or bandwidth from 150 to 3000 nm).

A photonic energy source which can be used in conjunction with the present invention can be selected from the group comprising: a laser, laser diode, light emitting diode (LED), arc flashlamp or a continuous wave bulb. The selection of the photonic energy source to be used in a particular embodiment of the present invention can be determined by the required spectral analysis. The functionality of the device may require a broad spectral analysis of the biological tissue or may require the spectral characteristics over a narrow bandwidth or even specific wavelength, for example.

For example, a laser has a very narrow spectrum (a highly coherent “single” wavelength), a narrow spatial beam, and high pulsed power. An incandescent light bulb has a broad spectrum, wide beam, and continuous transmission.

In one embodiment of the present invention, the electromagnetic radiation generated by the photonic energy source may be in the form of pulsed electromagnetic radiation.

Optical Emission Processing Means

The optical emission processing means receives light from the photonic energy source and delivers one or more illumination wavelengths in a pulse sequence to the biological tissue, wherein the optical emission processing means can comprise a means for isolating one or more illumination wavelengths and emitter optics that orient and focus the illumination wavelength(s) onto the biological tissue. The optical emission processing means is controlled by the emitter control electronics contained in the digital signal processing means, wherein the emitter control electronics may perform functions comprising pulse coding and pulse shaping, for example enabling the modulation of the illumination energy.

In order to distinguish the light wavelengths of reflection and fluorescence, which are received from the biological tissue, from ambient light noise, the illumination of the biological tissue should be performed using narrowband illumination.

In one embodiment of the present invention a generic device may require the ability to easily vary the emission spectral characteristics, such that spectral characteristics of the biological tissue can be determined for a range of illumination wavelengths. This can be accomplished by using a broadband light source, such as a halogen bulb or a Xenon tube and subsequently using wavelength separation optics to filter the emitted light thereby isolating narrow portions of the spectrum for illuminating the biological tissue. An alternate approach is to use an array of multiple narrowband or mediumband light sources (eg. laser diodes and/or various coloured LED's), each having particular desired spectral characteristics, and subsequently activate them one at a time, which effectively traverses a broad spectrum of light and isolates particular illumination wavelengths during the sequence of illumination of these devices.

The optical emission processing means may further comprise a light control device, which provides a means for modulating the light, which is to illuminate the biological tissue, for example producing a pulsed sequence of light emission. A light control device can be an indirect light modulator, for example, a light chopper, shutter, liquid crystal filter, galvanometric scanner or acousto-optic device. In addition, light modulation can be performed in a direct manner using an amplitude modulator circuit or a frequency modulator circuit. A worker skilled in the art would understand alternate method of modulating the illumination light emissions.

The wavelength separation optics associated with the optical emission processing means can be selected from fixed light conditioning optics including optical filters, refractive optics and diffractive optics and a variable light conditioning subsystem including a refractive or diffractive optical system whereby the optical centre wavelength is chosen by the use of a position controlled reflective surface after the light has passed fixed light conditioning optics or a refractive or diffractive optical system whereby the optical centre wavelength is chosen by use of a position controller to move fixed light conditioning optics. An example of a wavelength separation optic device is a monochromator. Other forms of wavelength separation optic devices would be known to a worker skilled in the art.

Emitter optics can be used to transmit the photonic energy between the components of the optical emission processing means and also to transmit the illumination light to the biological tissue. The emitter optics can be selected from the group comprising, condensers, focusing devices, fibre optics and apertures.

In one embodiment of the present invention, the optical system can include two monochromators: one monochromator which is controlled by said digital signal processing means to receive light from the illumination device and to deliver one or more wavelengths in a pulse sequence and a second a monochromator source which is controlled by said digital signal processing means to perform specific digital specific processing tasks to pass the one or more wavelengths' reactive characteristics at a specific time.

Received Light Optical Processing Means

The received light optical processing means collects and isolates one or more wavelengths of received light from the biological tissue, with this received light being related to the illumination of the biological tissue as described above. The received light optical processing means can comprise detector optics for collecting the received light from the biological tissue and a means for isolating one or more of the wavelengths of the received light for detection by the optical detector. The received light optical processing means is controlled by the emitter control electronics contained in the digital signal processing means and thus its function can be correlated with the optical emission processing means, which can provide a means for the efficient analysis of the received spectral emissions.

In one embodiment of the present invention, the received light optical processing means can isolate particular wavelengths of received light by using wavelength separation optics, which provides a means for isolating one or more wavelengths of received light thus allowing the received light to be correlated to the illumination wavelength.

The wavelength separation optics can be selected from fixed light conditioning optics including optical filters, refractive optics and diffractive optics and a variable light conditioning subsystem including a refractive or diffractive optical system whereby the optical centre wavelength is chosen by the use of a position controlled reflective surface after the light has passed fixed light conditioning optics or a refractive or diffractive optical system whereby the optical centre wavelength is chosen by use of a position controller to move fixed light conditioning optics. An example of a wavelength separation optic device is a monochromator. Other forms of wavelength separation optic devices would be known to a worker skilled in the art.

In a further embodiment of the present invention, the received optical processing means may be required to isolate one selected wavelength, for example, if the test specimen is illuminated by a particular wavelength of light and the reflection of this photonic energy by the biological tissue is required, the received optical processing means can have a fixed light separation means, since only a particular light wavelength is being evaluated.

Detector optics can be used to transmit the photonic energy between the components of the received light optical processing means and also to transmit the received light to the optical detector. The detector optics can be selected from the group comprising, condensers, focusing devices, fibre optics and apertures. In one embodiment of the invention an optical filter may provide this functionality, wherein the optical filter may include a low pass, high pass band filters or other compatible filters as would be known to a worker skilled in the art.

Optics Assembly

The optical system further comprises an optics assembly that provides a means for aligning the emitter optics of the optical emission processing means with the detector optics of the received light optical processing means. As such the orientation of the detector optics is directly related to the orientation of the emitter optics in addition to the location of the biological tissue to be scanned. For example, an optics assembly to be used for the in situ scanning of biological tissue can be configured in a different manner to an optics assembly for the scanning of an extracted test sample of biological tissue.

The orientation of the detector optics with respect to the emitter optics can be based upon the angle of reflection for example. The detection of the reflectance produced by biological tissue under examination can be enhanced by orienting the detector optics in the path of the reflected electromagnetic radiation upon its interaction with the biological tissue.

In one embodiment of the present invention the optics assembly is designed for in situ examination of biological tissue, for example skin tissue of a patient. In this configuration the optics assembly can be in the form of a probe which houses both the emitter optics and the detector optics, wherein the probe can be designed for hand held manipulation thereof or the probe can be supported by an adjustable arm enabling the correct positioning of the probe with respect to the insitu site of the biological tissue. Within this probe the detector optics are appropriately aligned with the emission optics.

In one embodiment of the present invention, the optics assembly is associated with a test sample housing which is used to retain the test sample of biological tissue in a desired orientation. In this embodiment, the optics assembly can be oriented with the test sample housing, since a biological tissue test sample upon placement within the test sample housing, can have the same orientation independent of the type of test sample. For example, as would be used with a microscope, the test sample housing may comprise a cover slip and a glass plate for securing the biological tissue test sample therebetween, and a set of clips may be used to orient, position and restrain the movement of this glass plate and the cover slip during testing of the biological tissue test sample.

Optical Detector

Each embodiment includes an optical detector which can sense the light transmitted by the received light optical processing means and convert this into an electrical signal for processing by the digital signal processing means and in particular the DSP received signal processing means.

A suitable optical detector can be a diode, photomultiplier, or a charge-coupled device (CCD) arranged in a linear array or an area array, for example. A specific example is a blue enhanced Gallium-Arsenide photodiode, a Cadmium Sulfide (CdS) photodiode or a silicon avalanche diode. Other suitable optical detectors would be readily understood by a worker skilled in the art.

Digital Signal Processing Means

Digital Signal Processing (DSP) means can be used to control the photonic energy source, the optical emission processing means and the received light optical processing means in order to be able to detect one or more wavelengths of the resultant radiation in relation to one or more wavelengths of illumination radiation, wherein this detection is being performed in the presence of noise introduced into the system. The digital signal processing means comprises emitter control electronics, which provide a means for controlling the illumination radiation (optical emission processing system) and the received light optical processing system. In addition, the DSP means comprises a received signal processing means which enables the DSP to correlate the received light radiation with the illumination radiation, which can provide a means for identifying reflectance, fluorescence and absorption from the biological tissue due to its illumination.

The emitter control electronics which control the illumination radiation performs tasks including: supplying electrical power and driving circuitry to convert electrical energy into light energy, controlling the amplitude and timing of light source pulses, controlling optical devices which filter, focus, or mechanically pulse the illumination radiation, for example, a light filter, monochromator, collimator and/or a chopper. In addition, the emitter control electronics provide a means for controlling the received light optical processing means enabling the isolation of reflectance and fluorescence light wavelengths from the biological tissue due to its illumination. For example, the incorporation of a monochromator into the received light optical processing means can provide a means for isolating desired wavelengths and the functionality of the monochromator is controlled by the received light optical processing system.

The coding function which is employed by the emitter control electronics in order to encode the illumination signal prior to interaction with the biological tissue can be provided by any number of signal modulation techniques. For example, pulse code software can be used to create a synchronous pulse for direct modulation of the light control device frequency (pulse frequency modulation, PFM). With PFM the frequency of the pulses is modulated in order to encode the desired information. Pulse code software can be used to create a synchronous pulse for direct modulation of the light control device amplitude (pulse amplitude modulation, PAM), wherein with PAM the amplitude of the pulses is modulated in order to encode the desired information. In addition, pulse code software can be used to create synchronous pulse for direct modulation of the light control device pulse width (pulse width modulation, PWM). With PWM the width of the pulses is modulated in order to encode the desired modulation. Finally the illumination signal may be encoded using a function generator to create a fixed synchronous pulse enabling pulse rate and amplitude modulation, in addition to a mechanical encoder driver to create a synchronous pulse for an indirect light modulator, for example a chopper, shutter, galvomirror etc.

In one embodiment of the invention the coding function which is employed by the emitter control electronics is binary phase shift keying (BPSK) which is a digital modulation format. BPSK is a modulation technique that can be extremely effective for the reception of weak signals. Using BPSK modulation, the phase of the carrier signal is shifted 180° in accordance with a digital bit stream. The digital coding scheme of BPSK is as follows, a “1” causes a phase transition of the carrier signal (180°) and a “0” does not does not produce a phase transition. Using this modulation technique a receiver performs a differentially coherent detection process in which the phase of each bit is compared to the phase of the preceding bit. Using BPSK modulation may produce an improved signal-to-noise advantage when compared other modulation techniques, for example on-off keying.

The DSP received signal processing means enables matched filter correlation between electrical signals received from the optical detector and the corresponding time period as defined by the emission control electronics. This correlation between transmitted and received signals can provide a means for enhanced identification of received signals over the noise (ambient light or electrical noise, for example) which may enter the optical system of the present invention. Filtering and time averaging of received signals, synchronized and matched with the emitted pulse sequence, enhances the signal-to-noise ratio (SNR) and improves the confidence in the measurement of the sample response at a wavelength or wavelengths of interest.

A matched filter is an exact copy of the signal of interest. The filter is correlated with the input signal, with this procedure basically being a sum of the products of the signal multiplied by the filter over the total duration of the filter. Upon the matching of the filter and the signal of interest, the correlation (convolution) sum typically peaks relative to the non-matched sums providing a means for identifying the signal over the external noise within the optical system. In one embodiment of the present invention, a bank of narrowband filters centered at intervals of the pulse rate can capture more lines from the pulse spectrum, and thus may provide a means for improved light pulse energy estimation and subsequent identification of the detected wavelength.

In one embodiment of the present invention, if the time domain spreading function is represented by F(ω) and the received signal is represented by H(ω), then the output of the matched filter receiver can be obtained using the digital signal processor:

$$s ( t ) = {\int }_{- ∞}^{+ ∞} F ( ω ) H ( ω ) {ⅇ}^{jω t} ⅆ f where \text{:} ω = 2 π f$$

In this equation F(ω) is the Fourier Transform of the input signal f(t) and H(ω) is the Fourier Transform of the receiver linear filter h(t). In a matched filter, the receiver linear filter H(ω) is adjusted to optimise the peak signal-to-noise ratio of the receiver output s(t) for a specific input signal f(t). When the receiver linear filter response H(ω) is given by:
H(ω)=KF*(ω)e^{-jax}^{₀}
then the output signal-to-noise ratio is maximised and the receiver filter response H(ω) is matched to the input signal f(t), wherein f(t) has the Fourier Transform F(ω). The two above equations are taken from “Information Transmission, Modulation and Noise, A Unified Approach to Communication Systems”; Schwartz, Mischa; Third Edition. A matched filter receiver enables one to potentially maximise the signal-to-noise ratio of the output signal s(t), the detection of which is desired. Thus a matched filter receiver may provide optimum detection of the output signal. Since a matched filter receiver is a linear system, s(t) is directly proportional to the intensity of the reflectance and fluorescence illumination on the detector. The use of a matched filter can enable one to detect weak signals in the presence of noise (external and internal noise of the optical system), which may not be detectable using other optical systems.

In one embodiment of the invention, the signal processing system involves both analog front-end and digital back-end tasks. In general the analog processing tasks are concerned with recovering the small sensor signals and applying highly selective filtering operations. The digital domain tasks are concerned with further signal filtering as well as analysis functions, in relation to energy detection and data output. To minimize the interference and to provide immunity against shot noise, the illumination signal is modulated by a frequency of typically a few hundred Hz. The analog section is designed to high gain amplify and prefilter the photodiode output and recover the modulation frequency. Utilizing these signals, a narrowband tracking filter can provide the very high selectivity for modulated signal recovery. The output of the narrowband filter, after amplification, is analog/digital converted and input into a DSP (digital signal processor) which in real time performs the back-end tasks of filtering, energy detection, averaging and converting the results into usable data. The filtering will further enhance the rejection of a/c noise and harmonic distortion, which may have been introduced in the final stages of analog processing. The filtering is followed by an averaging energy detector, which outputs the values proportional to the energy of the sensor signal. These values are sent to the host computer in short intervals, where they can be stored and processed for further analysis.

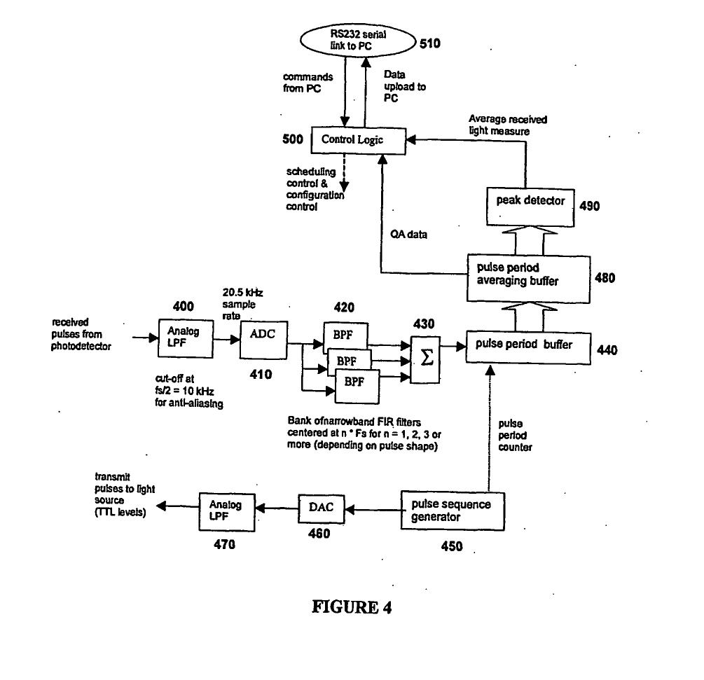

In another embodiment of the present invention, the digital signal processing means can be designed as illustrated in FIG. 4. Initially, a pulse sequence generator 450 transmits a pulse period counter to the pulse period buffer 440 and further transmits a digital signal defining the generated sequence to a digital to analog converter 460. The resulting analog pulses are sent to the light source upon passing through an analog low pass filter 470 and the light source subsequently illuminates the biological tissue based on these pulses. Upon the collection and detection of the emitted radiation from the biological tissue due to its illumination, the pulses generated by the photodetector as a result of photonic radiation detection are transmitted to an analog low pass filter (LPF) 400, which transmits the filtered information to a analog to digital converter (ADC) 410. The analog LPF can suppress frequencies over 10 kHz, for example, thereby providing anti-aliasing. This digitized information is sent to a bank of narrowband finite impulse response (FIR) filters 420, wherein each filter is matched to one of the lines in the pulse sequence spectrum (input signal pulse). This provides a means for matching the pulse spectrum in order to identify the signal over the external noise within the system (match filtering). The sums of the filter—input signal correlation 430 are transmitted to the peak detector through pulse period buffers 440 and 480 and the average light measured is then sent to the control logic 500 of the DSP. The control logic 500 provides a means to perform scheduling control and configuration control of the digital signal processing (DSP) means. The averaged measured light signals are subsequently transmitted to a computing device located on a personal computer, for example, via a RS232 serial port 510, in order to be organised into a usable and presentable format, for example generating a graphical representation of the collected information.

The utilization of advanced signal processing techniques, enables the detection of optical reflectance and fluorescence emissions that may normally not be detected.

Moreover, the signal processing algorithms can be implemented in standard digital signal processing chips, enabling the overall cost of devices based on this technology to be relatively low.

The DSP means can be incorporated into a computer system in the form of a circuit board that can be installed in a computer, wherein the computer can provide a means for manipulating and organising the received information after matched filtering into a format that is easy to interpret by the operators of the system, for example. Alternately, the DSP may comprise stand alone hardware providing a means for the DSP to function independently of a computing device, wherein scanning results can be transmitted to a computing device after data analysis for the performance of further operations comprising, organisation, display and storage of the information, for example.

Stand Alone DSP System

In this embodiment the stand alone DSP associated with the optical system comprises a transmitter and receiver block, a micro-controller block (MCU), a networking block and a digital and analog power supply block.

In this embodiment the DSP block comprises a digital signal processing chip and an additional external static random access memory (SRAM). The DSP block performs the computation algorithms for fast, real-time processing of spectral data being transferred from the optical detector(s). This block also generates signals that are capable of modulating the photon energy source, wherein this modulation signal can be multiplexed to multiple photon energy sources if required. However, each detector, if there is more than one, has a separate channel into the DSP block for the transmission of information relating to the received light. In addition, the DSP block can control the optical device(s) that mechanically pulse the illumination radiation, for example, a chopper. As would be known to a worker skilled in the art, the required processing speed of the DSP chip can be determined by the estimated amount and frequency of the incoming data that is to be processed, for example. In this manner an appropriate chip can be determined based on its processing speed for example the number of hertz that the DSP operates, 40 Hz, 60 Hz and so on.

According to this embodiment, the transmitter and receiver block comprise analog-to-digital converter(s) (ADC), digital-to-analog converter(s) (DAC) and low-pass filters, wherein these filters enable anti-aliasing of the received signal. If light emitting diodes (LEDs) or laser diodes are used as the photon energy source for the optical system, this block further comprises a multiplexer and high current amplifiers. The multiplexer enables the transmission of signals for the activation of the multiple photon energy sources independently and the high current amplifiers provide a means for providing sufficient energy in order to activate these photon energy sources such that their maximum spectral power output is obtained.

The networking block of the stand alone DSP means comprises a networking card, for example, an ethernet chip or a wireless network chip, which enables the interconnection of the stand alone DSP system to a communication network, for example a local area network (LAN), a wide area network (WAN) or a wireless network (for example Bluetooth™ or IEEE 802.11). A worker skilled in the art would understand the format and type of chip or card that is required for the desired network connection. In addition the network block further comprises a serial interface chip, for example a RS-232 port which can provide a serial interface to another component or system, for example a computer or a serial modem for example a dial-up or wireless type modem. This type of interconnection can provide a single computing device the ability to collect data from a plurality of optical systems for example, thereby centralising the data collection site.

Furthermore, in this stand alone embodiment, the micro-controller unit (MCU) block comprises a MCU chip, which may be an 8-bit, 16-bit or 32-bit chip, for example, an external SRAM and an external FLASH unit. The MCU block manages the DSP block and the networking block, wherein the MCU block collects processed data from the DSP block and forwards this information to the networking block. Optical devices which filter and/or focus the illumination radiation and received light, for example light filters or monochromators, can be controlled by the MCU block. The MCU block may additionally perform statistical analyses on the data and may possibly activate an alarm setting. For example, an alarm setting may be activated if the level of fluorescence of the biological tissue exceeds a predetermined level, wherein this alarm activation may comprise the collecting of a sample for a more detailed analysis or the notification of personnel of the alarm activation. In the case where software updates to the DSP block are required, for example the modification of the match filtering procedure, the MCU block can manage the remote software updates of the DSP code, for example. The type of MCU chip incorporated into the MCU block may vary depending on the volume of information that is to be processed for example, as would be known to a worker skilled in the art.

The digital and analog power supply block of the stand alone DSP system can provide regulated DC power at a variety of levels depending on that required by the components of the stand alone DSP system. In one example, the input power to this stand alone system may be supplied by an unregulated or varying power supply, for example a wall plug. The digital and analog power supply block comprises elements which can regulate the input power and subsequently generate the required analog and digital voltage levels for each component of the stand alone DSP system. As examples, elements which enable the adjustment of the input power comprises transformers, AC-DC converters or any other power regulation element as would be known to a worker skilled in the art.

Pattern Generation Using the Optical System

The optical system according to the present invention is capable of illuminating biological tissue with a predetermined wavelength of electromagnetic radiation and subsequently scan the resulting emitted electromagnetic radiation from the illuminated biological tissue, wherein this scanning is performed for a plurality of wavelengths. This scanned plurality of wavelengths of emitted electromagnetic radiation are wavelengths that are greater than or equal to the illumination wavelength. Emitted electromagnetic radiation having a wavelength equal to that of the illumination wavelength represents reflected energy and the longer wavelengths represent fluorescence of the biological tissue due to its illumination. In one embodiment of the present invention, this scanning ability of the optical system can be provided by a dual light separation system wherein a first monochromator is electronically controlled to select the illumination wavelength and a second monochromator is electronically controlled to select the reflectance and fluorescence emission wavelengths emitted from the biological tissue due to its illumination by the selected illumination wavelength that are to be scanned.

In one embodiment of the present invention, the procedure for the generation of a pattern of optical characteristics of biological tissue can be provided by illuminating the biological tissue with one or more encoded and predetermined wavelengths of electromagnetic radiation, in order to generate encoded reflectance and fluorescence from the biological tissue. Subsequently, collecting this encoded reflectance and fluorescence associated with the predetermined wavelengths of illumination electromagnetic radiation and then decoding the generated encoded reflectance and fluorescence associated with the predetermined one or more wavelengths. This procedure enables the identification of the reflectance and fluorescence, over the noise within the system, being collected from the biological tissue for a particular illumination wavelength. These steps are performed a number of times in order to allow the collection of the reflectance and fluorescence properties of the biological tissue for other illumination wavelengths. Upon the collection this data and correlating the reflectance and fluorescence with a particular illumination wavelength, this information can be plotted as a contour map wherein the x and y coordinates of the map represent the illumination wavelength and the detected emission wavelengths and the contours represent the intensity of the detected emitted electromagnetic radiation. In this manner a pattern of the reflectance and fluorescence of biological tissue due to its illumination can be determined.

In one embodiment of the present invention, a contour map representing the difference in the intensity of the detected electromagnetic radiation between two different biological tissue samples can be created. In this manner one is able to identify a pattern of difference therebetween. For example, one can evaluate the spectral differences between biological tissue from two different sources or normal versus abnormal biological tissue from the same source. Other comparisons are possible as would be readily understood by a worker skilled in the art.

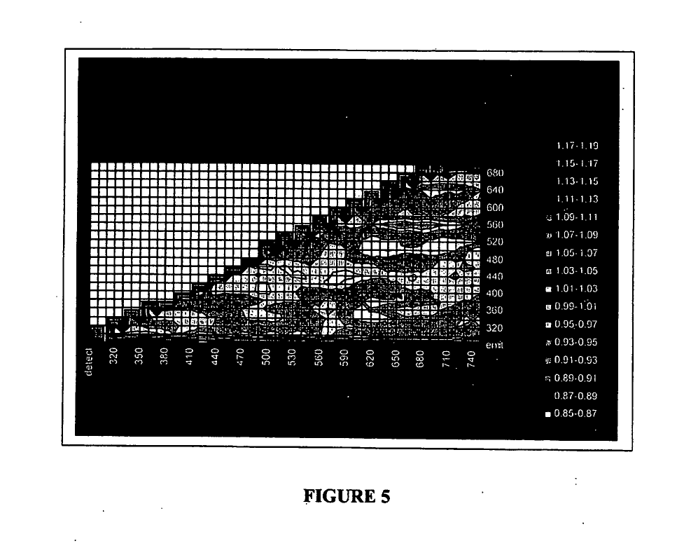

For example, FIG. 5 illustrates a contour map representing the difference between the spectral signatures of a tumorous liver tissue sample with respect to a normal liver tissue sample, wherein these samples are taken from the same individual. Having specific regard to this figure, one can identify significant differences in the reflectance thereof, which is represented by the square areas along the diagonal. In addition, an increase of approximately 3% in the fluorescence of the tumorous liver tissue sample with repsect the normal liver tissue sample can be identified for an illumination wavelength (emit) of 440 nm and a detected wavelength range between 570 and 610 nm. Finally, a decrease in fluorescence can be identified for an illumination wavelength of 560 nm and a detection wavelength ranging between 640 and 700 nm.

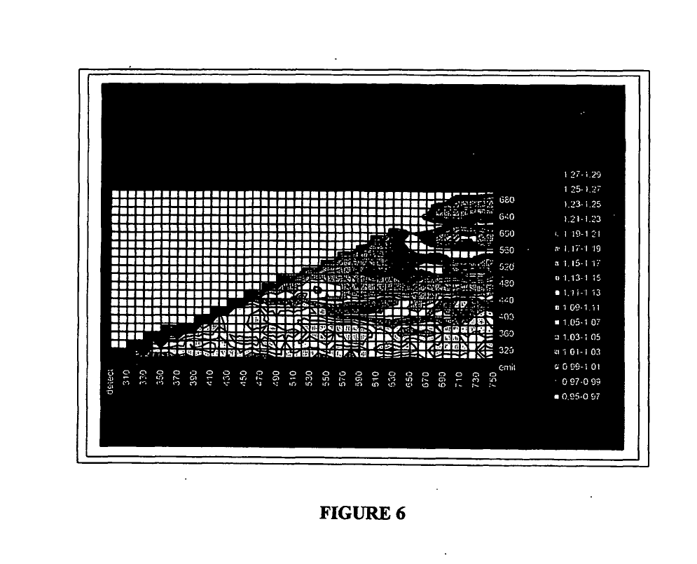

Having regard to FIG. 6, a contour map representing the difference between the spectral signatures of a liver tissue sample from a first individual with respect to a liver tissue sample from a second individual is provided. In this figure one is able to identify that the emitted spectral characteristics of the liver tissue samples are essentially the same for illumination wavelengths of 480 nm and greater. However, the fluorescence of the liver tissue samples differ by approximately 14% for illumination wavelengths between 340-380 nm and detected fluorescence wavelengths between 520 and 600 nm.

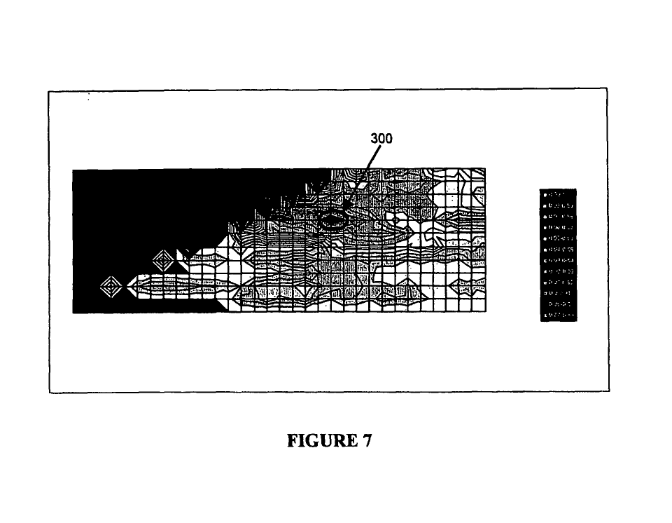

In addition, having regard to FIG. 7, a contour map representing the difference between the spectral signatures of a tumorous skin tissue sample with respect to a normal skin tissue sample from the same individual is provided. As identified by 300 a decrease in the fluorescence of approximately 10% is identified between the tumorous skin tissue sample with respect to the normal skin tissue sample.

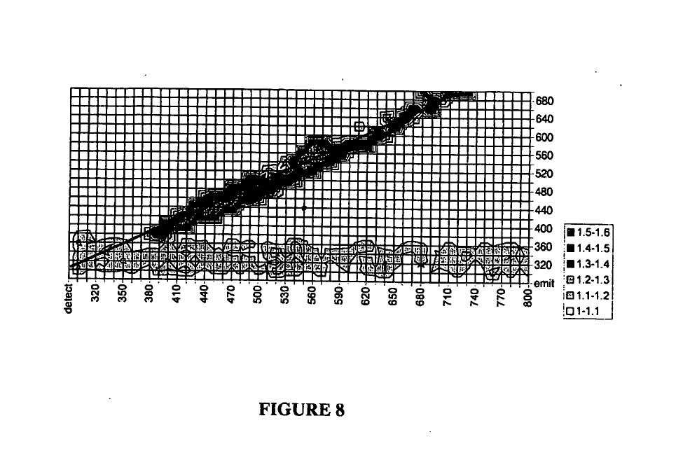

With reference to FIG. 8, a contour map is illustrated that represents the difference between the spectral characteristics of a bronchogenic malignant tumour tissue sample with respect to a normal tissue sample in close proximity after data reduction using a high pass digital signal filter is performed. From this figure, one can identify the increased reflectance of the tumorous tissue with respect to the normal tissue, which is noted along the diagonal, in addition to the fluorescence difference for illumination wavelengths below 400 nm.

In one embodiment of the present invention, a three dimensional surface plot can be created in order to provide a more graphical representation of a pattern detected by the optical system, as opposed to a contour map. A three dimensional representation can provide a means for more easily identifying the variations in the intensity of the detected electromagnetic radiation from the biological tissue based on its illumination.

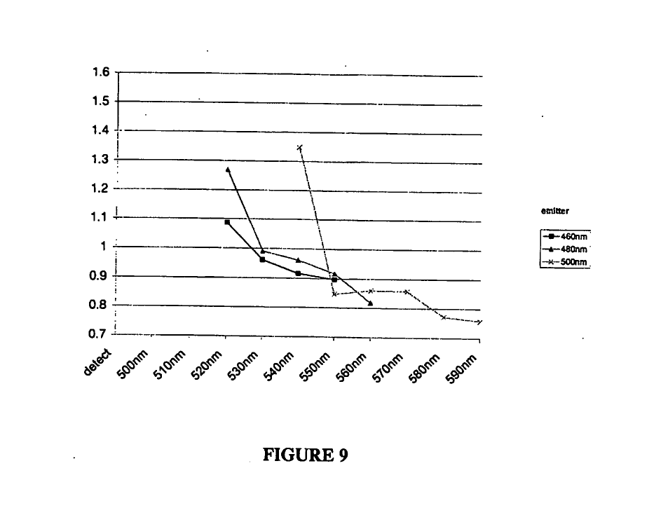

Using the optical system of the present invention, other types of patterns of the optical characteristics of biological tissue can be determined. For example, with reference to FIG. 9, a pattern representing the drop in the relative intensity in fluorescence of a tumorous tissue sample with respect to a norml tissue sample for three different illumination wavelengths is illustrated.

It has been suggested in the prior art, for example by Douglas et al, “Characterization of the Autofluorescence of Polymorphonuclear Leukocytes, Momonuclear Leukocytes and Cervical Epithelial Cancer Cells for Improved Spectroscopic Discrimination of Inflammation from Dysplasia”, Photochemistry and Photobiology, Vol. 71, Issue 3, that the identification and scanning of particular biomarkers, for example NAD(P)H, can provide a means for evaluating tissue samples.

As such, an alternate method of identifying a pattern in the optical parameters of a first type of tissue with respect to a second type of tissue, can be provided by identifying and scanning for one or more particular biomarkers within the tissue samples. For example, the optical system was used to evaluate the level of fluorescence of three particular biomarkers, namely nadh, flavins and tryptophan and these fluorescence levels were correlated between a bronchogenic malignant tumour tissue sample and a normal tissue sample. Having specific regard to NADH, this biomarker was illuminated by energy having a wavelength of 380 nm and the fluorescence emitted thereby at a wavelength of 440 nm was detected. Prior art studies suggest that the level of fluorescence emitted by nadh in the tumorous tissue sample should be less than that of the normal tissue. The optical system according to the present invention identified a 15% drop in the fluorescence. Likewise having regard to the flavins biomarker, an illumination wavelength of 450 nm was used and a fluorescence wavelength of 515 nm was scanned. Prior art studies suggest a decrease in the level of fluorescence, and the optical system of the present invention identified a 3% decrease in the fluorescence of this biomarker, when comparing the tumorous tissue sample with the normal tissue sample. Finally, a comparison of the level of fluorescence of tryptophan between a tumorous tissue sample and a normal tissue sample was determined. The tissue samples were illuminated with energy having a wavelength of 300 nm and fluroescence at a wavelength of 350 nm was scanned. An increase of 10% in the level of fluorescence was detected by the optical system according to the present invention, wherein the prior art suggests that there should be an increase or no change in this characteristic of the biomarker, when comparing the tumorous tissue sample with the normal tissue sample.

Scanning Methodologies

For manual scanning, an optical probe can be moved manually across the surface to be analyzed such that it analyzes only the area immediately under observation. The spectral characteristics can be observed at a fixed point in space (xₒ, yₒ) and as such, one obtains a one-dimensional plot of the spectral response for each point (xₒ, yₒ). This mode of scanning can be useful if the fluorescing material is diffusely distributed throughout the medium to be observed, or if localized analysis is required.

For two-dimensional scanning, an optical probe can be moved (or scanned in some other manner) across a two dimensional surface and spectral responses can be obtained for each point (xᵢ, y_{I}, λ) in the plane. This method represents an analysis of a sample in three dimensions, that is (xᵢ, yᵢ, λ), wherein this mode of operation can be useful if the fluorescing material is highly localized within a larger area of observation.

Finally, for three-dimensional scanning, quantitative and qualitative data can be obtained for closed loop feedback control and detection of physical and optical characteristics in the biological tissue under examination. The probe can be scanned across a two-dimensional surface such that spectral responses can be obtained for each point represented by (xᵢ, y_{I}, z₁, λ).

Considerations

In one embodiment, the requirements of the optical system are that: 1) it is able to resolve optical spectra over the range 250 nm to 800 nm; 2) the spectral resolution is on the order of 5 nm of better; and 3) that it has a stray light suppression of 10⁻⁵ or better, for both the illumination and emission units. In addition, a spectral resolution of 5 to 10 nm can allow reasonable sampling of the fluorescence peaks, which appear to be the order of 30 to 50 nm. However, finer resolution may be useful in some applications. The stray light supression factor required depends on how samll an area of received light one wishes to detect. Stray light essentially determines the optical noise floor for the system, and sets the limit of optical detectability.

In choosing the illumination wavelength, the factors that should be balanced are overall scanning time for the area of interest and the resolution of the scan. The total number of steps N required to sweep out the emission and fluorescence spectrum is;
N=nᵢ·n_{d}/2
where:
nᵢ=number of steps for the illumination monochromator
n_{d}=number of steps for the reflection/fluorescence monochromator

The factor ½ determines that only the diagonal terms of the emission/fluorescence matrix are of interest and terms on one side of the diagonal. Moreover, N is proportional to Δλ/2, where Δλ is the spectral resolution of a monochromator. Since the scanning time is proportional to N, then there is a trade-off between Δλ and the scanning time.

Weak Signal Detection

In one embodiment, the tone encoded method is used for signal encoding due to its basic simplicity, and the fact that it yields a reasonable degree of noise suppression relative to complexity. In this embodiment, the key consideration is the amount of time required to take one measurement. This is determined by: 1) the amount of time required to acquire the samples for a frequency domain transfer, which is essentially the number of samples required divided by the sample rate; and 2) the filter bandwidth in the case of a bandpass filter technique, which is essentially the reciprocal of the bandwidth of the filter.

The trade-off with the electrical signal bandwidth is observation time versus noise. As the bandwidth is increased and the observation time decreased, the noise power increases in proportion to the bandwidth. Any increase in noise can reduce the detector sensitivity. The total processing time to scan the area of interest is simply T=Nτ, where τ is the time for one measurement at one wavelength. The two key variables in the observation time are the optical filter bandwidth and the electrical filter bandwidth.

A rough first order calculation of T can be made by making the following assumptions: 1) resolve optical spectra over the range 250 nm to 800 nm; 2) use an optical resolution bandwidth of 10 nm; and 3) use an electrical bandpass filter BW of 10 Hz, therefore τ=0.10 sec. By using these assumptions, the scanning time is 151.25 seconds, or about 2.5 minutes.

When biological tissue to be examined is exposed to illumination radiation, the detection of its reactive radiation characteristics is the goal. However, in general, the fluorescent light will be much weaker than reflected light from the biological tissue due to its illumination. The spectral resolution required is determined by the ability of the optical system to discriminate between reflected and fluorescent wavelengths. This can be achieved through the use of prism and/or grating monochromators with variable apertures, for example, which suppress stray radiation.

For optical signatures to be adequately resolved, the system should be able to detect very weak electrical signals, which result from the optical radiation being detected by the photodiode. Ultimately, the goal is to detect a very weak signal in a background of noise due to electrical noise, optical background radiation, and out of band emissions from the biological tissue.

Other variables in the measurement of optical patterns of biological tissue comprise: a) time duration the biological tissue is illuminated; b) the amplitude of the illumination at the biological tissue's first surface; c) the amplitude of the noise variables; d) spectral shifts in the illuminators over time; and e) the decay of the fluorescence emitted by the biological tissue after the illumination of thereof has been discontinued. These variables need to be addressed in order to compare the performance of various detection schemes.

In one embodiment of the present invention, adaptive filtering of the received light may enable the detection of the decaying intensity of fluorescence emitted from the biological tissue upon the discontinuation of the illumination thereof. The discontinuation of the illumination may be complete termination of transmission of photonic energy or the discontinuation of a particular illumination wavelength.

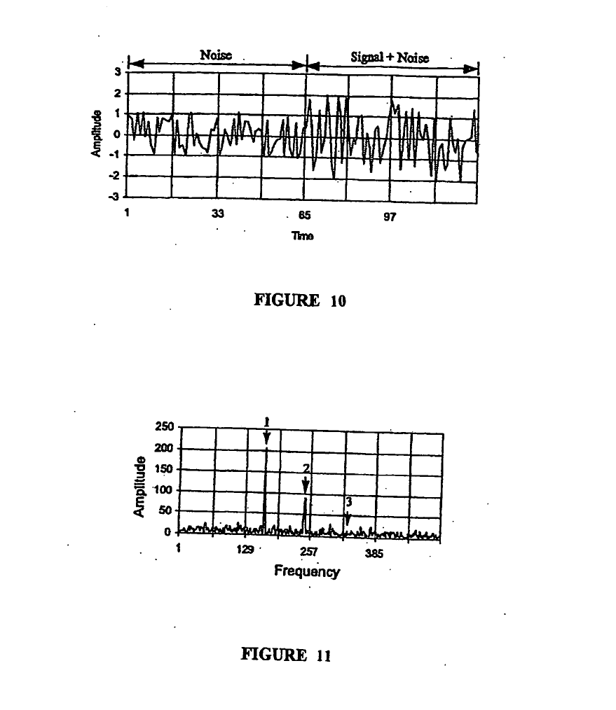

For example, pulse amplitude modulation techniques as applied to this situation can be essentially On-Off keying of the illumination. The detection is based on the ability to detect the presence of the signal in an ambient noise. The signal detectability depends on the ability to discriminate the signal from the noise, and generally requires a signal power much greater than the noise (>10 dB typically). An example of an On-Off keyed signal is shown in FIG. 10. The signal to noise ratio (SNR) in this case is 0 dB, and it is not possible to distinguish the noise portion of the signal from that consisting of signal plus noise.

The frequency domain detection mechanism is simply a detection means based on frequency modulation of the signal with a constant frequency modulation. This has advantages over time domain detection means such as On-Off keying. Even though the RMS amplitudes of the signal and the noise can be equal (SNR=0 dB), the power spectral density of the modulated signal is usually greater than the power spectral density of the broadband noise. The carrier can be isolated from the noise by a number of means, including: a) spectral measurement techniques, such as a DFT or FFT; and b) narrow band filtering with the centre frequency of the filter located at the modulation frequency.

An example of this is shown in FIG. 11. In this case, the RMS amplitudes of the first signal and the noise are equal (SNR=0 dB). Two other signals were added which had magnitudes relative to the first signal of 0.50 and 0.1 respectively. The time domain signal happens to look exactly like that shown in FIG. 10. In the frequency domain however, the spectral peaks for the first and second signals are apparent. The spectral signature for the third signal however is buried in the noise and cannot be resolved. This detection technique is relatively simple to implement in practice, and is suitable for use in the optical system of the present invention.

The pulse coding techniques (binary, linear, enhanced) are an alternative means of detection. Pulse coding techniques are often used to detect very weak signals in the presence of noise. They are more complex than traditional techniques such as tone detection and pulse amplitude detection, however they are sometimes the only choice when the amplitude of the signal to be detected is weak relative to the noise and there are no means available to increase the signal to noise ratio other than pulse coding. Two pulse coding techniques are Binary Pulse Coding and Linear Frequency Modulation (FM) Coding. Both of these techniques fall into the realm of pulse compression and spread spectrum, and they are adequately described in numerous references, for example Barton, DK (1978) Radars Volume 3: Pulse Compression, Artech House Inc.

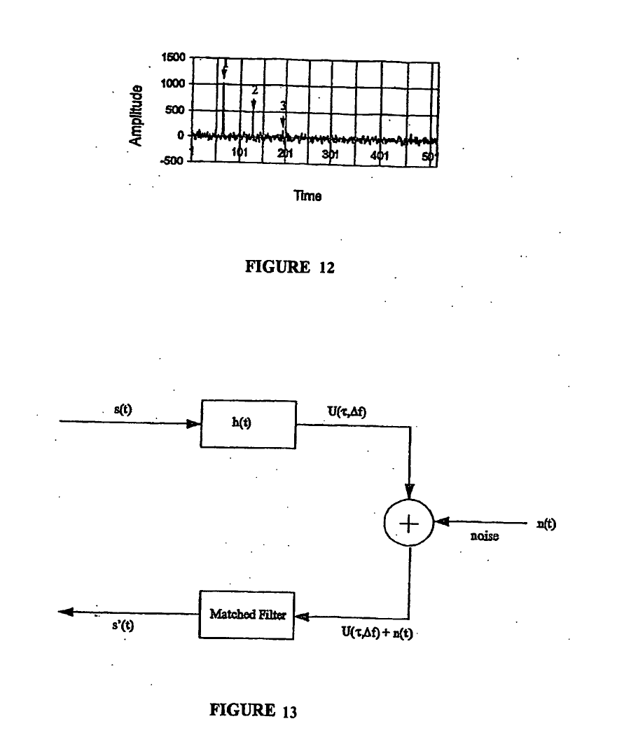

Binary Pulse Coding, as an example, uses a 1000-bit synchword, which can be created, by using a uniform random number generator and constructing a binary sequence from that data. Pulses are generated at specific locations in the time domain and the relative amplitudes are measured. Results of a time domain correlation output are shown in FIG. 12. In an amplitude plot, all three pulses can be detected. The third and smallest signal pulse is just distinguishable from the noise.

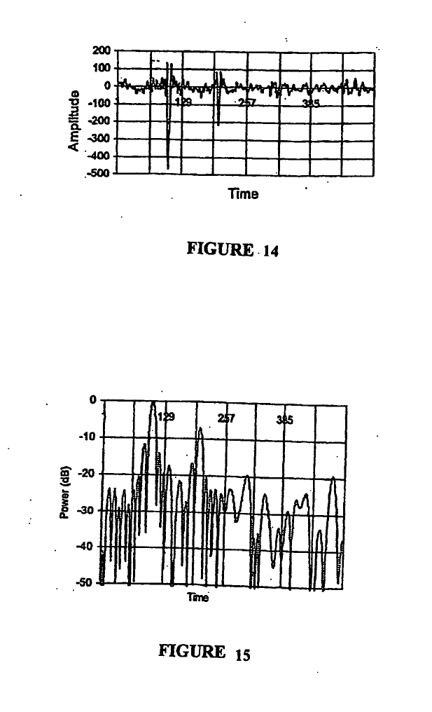

Linear FM Pulse Compression schemes have traditionally been used in radar systems to reduce the overall peak power of transmitted signal while still achieving large detection ranges. They also figure prominently in Synthetic Aperture Radar processing for airborne and spaceborne imaging radars. This form of coding is achieved by linearly sweeping a carrier signal from f₁ to f₂ (for a swept bandwidth of Δf) for a duration τ. In general, the “output power” of a linear FM coded signal is increased by the Time Bandwidth Product (TBP) Δfτ, which is the product of the pulse duration in seconds and the swept bandwidth in Hertz. The detection process is essentially a matched filter detector, which is matched to the linear FM transmitted pulse. The overall process is shown in FIG. 13. The signal s(t) is usually a Dirac Delta function, which in reality is simply the trigger pulse for the encoder h(t) which generates the transmitted signal U(τ,Δf) which is the linear FM coded pulse (or Chirp) which has a duration τ and a bandwidth Δf. This is the signal that would drive an optical emitter to illuminate a subject. Noise n(t) is added to the coded signal in both the optics and the electronics. This optical signal is detected by a photo detector, whose electrical output signal is comprised of the actual optical signal of interest, optical background noise, and electrical noise in the photo detector and electronics. The matched filter detector then processes this electrical signal. Since the optical signal of interest is the only one of the three components of the signal, which is matched to the matched filter, it is the only component which experiences gain due to the linear FM pulse coding. The optical and electrical noise components are essentially suppressed relative to the coded signal. This is the key advantage of such a scheme. A linear FM Chirp output is shown in FIG. 14. In the amplitude plot, only the largest two pulses can be detected, with the third being buried in the noise and it is arguable if it is visible or not. This example graphically demonstrates the coding gain offered by a linear FM Pulse Compression Technique.

Enhanced Pulse Coding Techniques take advantage of the fact by increasing the Time Bandwidth Product, greater coding gain can be achieved. Using this technique the weakest of the time domain pulses was just visible.

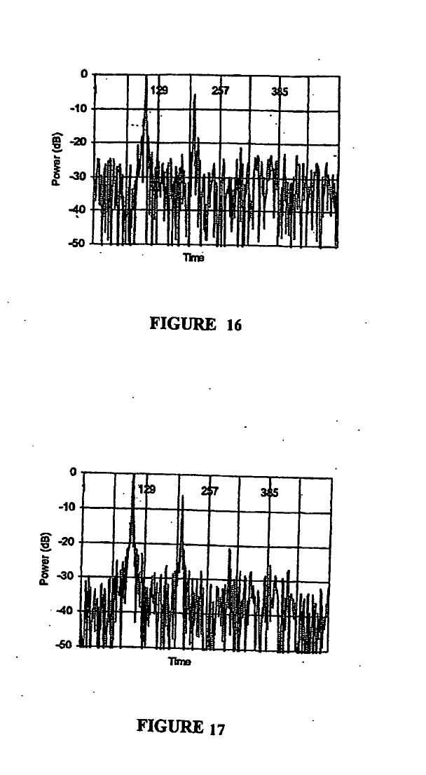

A plot of the original case with a TBP of 200 is shown in FIG. 15 and the new case with a TBP of 800 is shown in FIG. 16. The increase of the time bandwidth product has increased the coding gain sufficiently enough that the third and weakest pulse is now visible above the noise floor. The coding gain was increased from 23.0 dB to 29.0 dB, or an overall increase 6.0 dB. In both plots, the power has been normalised to the peak located at sample 100. The drop in the noise floor in going from a TBP of 200 to 800 is readily apparent.

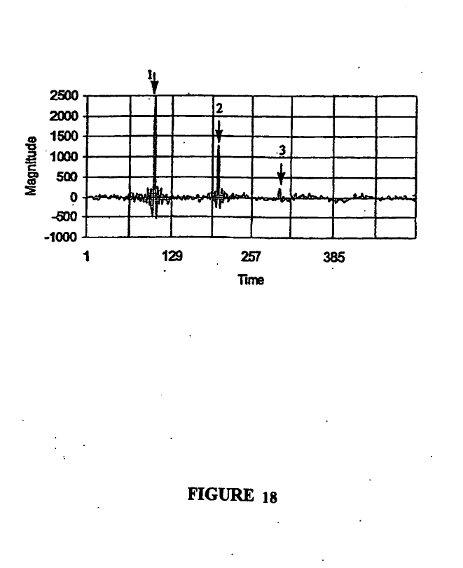

To further make this point, plot for the case of a TBP of 2250 is shown in FIG. 17. In order to compare this high time bandwidth product detection scheme to the other coding techniques, a time domain magnitude plot where the detector amplitude has been plotted is shown in FIG. 18. The noise amplitude should be suppressed by √2250, or about 47.4. The peak amplitude of pulse 1 is 2505, pulse 2 is 1252, and pulse 3 is 250. The noise magnitude was the same as that for the signal for peak 1, therefore the noise magnitude should be suppressed to a level of approximately 52. As seen from FIG. 18, this is, more or less, the case. Due to the high level of noise suppression achieved, the signal for pulse 3 is visible relative to the noise background. This is readily apparent when the TBP=375 case in FIG. 14 where pulse 3 is not visible is compared with the TBP=2250 case in FIG. 18 where pulse 3 is readily visible.

Higher Time Bandwidth Products can be used to achieve higher coding gains, however these may be limited depending on the means used to achieve the signal coding. A mechanical chopper would be limited by the ability to replicate the linear FM code onto the chopper wheel, whereas acoustic-optic modulators could achieve much higher TBP's but at much higher expense.

To gain a better understanding of the invention described herein, the following examples are set forth. It should be understood that these examples are for illustrative purposes only. Therefore, they should not limit the scope of this invention in any way.

### EXAMPLES

### Example I

### Spectrometer Incorporating a Matched Filter Receiver

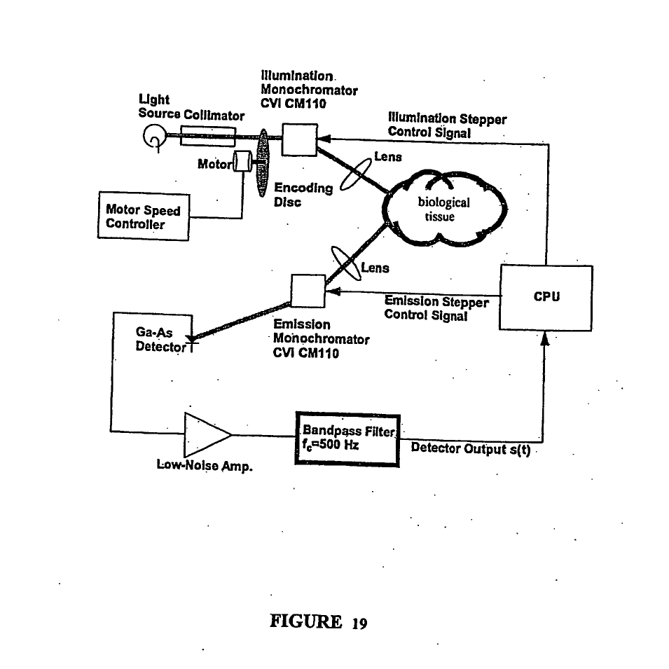

One embodiment of the present invention is shown in FIG. 19 and comprises a light source, for example, a miniature Xenon bulb that has an emission spectrum approximately equal to that of a 6000 ° K Blackbody with a few discrete spectral lines. The light is collimated and modulated by a chopper wheel, which provides a 500 Hz On-Off modulation to the light entering the Illumination Monochromator. The Illumination Monochromator operating under the control of the CPU sweeps the illumination wavelength from 250 nm to 800 nm in steps of 10 nm. This illumination is focused onto the Area of Interest, of the biological tissue. The Emission Monochromator operating under the control of the CPU sweeps the illumination wavelength from 250 nm to 800 nm in steps of 10 nm. It is controlled in such a way that for every illumination wavelength sample λᵢ, it sweeps over the range of wavelengths greater than or equal to λᵢ. A Ga—As photodiode is used as the optical detector, with the signal from the photodiode being amplified by a Low Noise Amplifier (LNA). The output of the LNA can be filtered using an analog filter, or it can be digitized using an Analog to Digital Converter (ADC) and processed digitally using an IIR or FIR digital filter. The detector output is recorded for each λᵢ and λₑ, and can be plotted for display based on coordinates defined based on λᵢ and λₑ and further defined by the intensity of the detected electromagnetic radiation. The type of plot can be presented as a contour map or alternatively can be presented as a three dimensional surface plot, for example. This type of pattern generation for the collected optical parameters, can allow comparison of samples of biological tissue, for example from different sites or biological entities or normal biological tissue with abnormal biological tissue.

A issue to be dealt with for this example of the optical system, is the magnitude versus wavelength calibration of the system, since the Xenon Light source is not spectrally flat. This issue can be can be compensated for by using a standard diffuse reflection source, which is spectrally flat in an optical wavelength sense. A calibration factor can then be applied to the collected data such that the spectral colouring of the illumination source can be removed from the data. This process of correction can be seen essentially as spectral equalization of the data.

Once the data is equalized, it can be displayed in a number of ways such as contour plots, surface plots, etc. for easy visualization of the illumination/emission spectra, thus creating a optical pattern for the biological tissue under examination. This procedure of display may require normalization of the collected data to for example the strongest spectral peak of A response at a fixed wavelength location, for example, wherein this type of correlation can be determined experimentally.

### Example II

One embodiment of this invention comprises an optical system comprising a spectrometer, an electronic light modulator and digital signal processing means, including: a) a light emitting diode (LED), as the illumination light source, which is controlled by said digital signal processing means to emit a radiation bandwidth ranging from 380 to 500 nanometers; b) a stepper motor controlled, grating monochromator which is controlled by said digital signal processing means to receive light from the illumination device and to deliver the N^{th} wavelength in a pulse sequence; c) an optical fibre probe that is coupled to the monochromator with collimating and focusing elements that delivers the N^{th} wavelength to the biological tissue, located in an assembly that orients the illumination optics with that of the collecting optics such that they are at a constant angle to each other; d) collecting means for gathering the resultant radiation of the N^{th} wavelengths and delivering the information via light collection lenses and fibre coupled to the stepper motor controlled, grating detection monochromator; and e) a photodetector such as a Ga—As Integrated Photodiode and Amplifier. The stepper motor controlled, grating monochromators are controlled by said digital signal processing means to perform tasks to pass the N^{th} wavelength reactive characteristics at a specific time.

Typically a Ga—As Integrated Photodiode and Amplifier is made up of stock electronic components that consist of a photodiode and transimpedance amplifier on the same chip. This is sampled by the digital signal processing means to sense the radiation at a specific time. A photodiode is used as an optical detector, with the signal from the photodiode being amplified by a Low Noise Amplifier (LNA). The output of the LNA is filtered using an analog filter to condition the signal from the photodetector with an op amp amplifying the signal to a specific range and digitised using an Analog to Digital Converter (ADC) and processed digitally using an FIR digital filter and a digital signal coding software technique such that a time/bandwidth product can be measured using a correlation receiver.

The system further comprises a DSPS device where an illumination modulation coding signal is created using a 32 bit linear FM pulse coding technique for pulse compression, the detection pulse coding is resolving the time bandwidth product with a matched correlation receiver, and the detection of specific amplitudes of irradiance can prompt the DSPS to run a specific routine to test for specific signal response characteristics in this case fluorescence and reflectance can be measured depending on the limitations of the wavelengths of illumination. The monochromator gratings can operate through the visible spectrum and can be substituted for other wavelengths into the UV or IR; and a digital signal processing technique such that software that recognises the peaks of data and their rule based weighted relevance can control the illuminator and detector monochromators.

### Example III

In one embodiment of the present invention an optical system can be designed with the ability to control the wavelength of the scan (illumination radiation) including modulation techniques. This type of optical system can provide maximum optical flexibility in relation to research and diagnostic applications.

An embodiment of the optical system designed for this scenario comprises: a digital signal processing means which is integrated into a computing device with the emitter control electronics comprising pulse code software to create a synchronous pulse for direct modulation of the optical emission processing means frequency and the received signal processing means incorporating a signal correlation match filter; a flashlamp providing the photonic energy source; optical emission processing means incorporating a frequency modulator circuit for modulating the illumination radiation, a refractive or diffractive optical system whereby the optical centre wavelength is chosen by the use of a position controller to move the fixed light conditioning optics of the emitter optical system; received light optical processing means incorporating a refractive or diffractive optical system whereby the optical centre wavelength is chosen by the use of a position controller to move the fixed light conditioning optics of the detector optical system; and a silicon APD photodetector acting as the optical detector.

### Example IV

In one embodiment of the present invention an optical system can be designed for maximum sensitivity of resultant radiation resulting from the illumination of the biological tissue by a known wavelength of light. This type of optical system can be useful for fluorescence analysis, especially if a spectral probe is attached to the subject of interest and has know spectral properties such that detection of a specific wavelength of fluorescence, absorption or reflection can be measured.

An embodiment of the optical system designed for this scenario comprises: a digital signal processing means which is integrated into a computing device with the emitter control electronics comprising pulse code software to create a synchronous pulse for direct modulation of the optical emission processing means frequency and the received signal processing means incorporating a signal correlation match filter; a laser providing the photonic energy source; optical emission processing means incorporating an acousto-optic scanner and a fixed emitter optical system; received light optical processing means incorporating fixed light conditioning optics; and a photomultiplier acting as the optical detector.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. An optical system for detecting one or more optical responses of biological tissue, said optical system comprising:
(a) a photonic energy source for emitting electromagnetic radiation;
(b) an optical emission processing means for receiving the electromagnetic radiation from the photonic energy source and isolating one or more illumination wavelengths of the electromagnetic radiation, said optical emission processing means encoding the one or more illumination wavelengths using one or more pseudo random codewords or linear FM thereby generating an encoded signal, the optical emission processing means transmitting the encoded signal to the biological tissue;
(c) an optics assembly providing a means for aligning emitter optics of the optical emission processing means with detector optics of a received light optical processing means;
(d) a received light optical processing means for collecting and isolating one or more wavelengths of received electromagnetic radiation from the biological tissue created in response to the encoded signal and transmitting the one or more wavelengths of received electromagnetic radiation to an optical detector;
(e) an optical detector for sensing and converting the one or more wavelengths of received electromagnetic radiation into an electrical signal; and
(f) digital signal processing means for correlating the electrical signal received from the optical detector with the encoded signal thereby identifying an optical response of the biological tissue to the one or more illumination wavelengths, said digital signal processing means controlling the functionality of the photonic energy source, the optical emission processing means and the received light optical processing means.

2. The system for detecting optical characteristics of biological tissue according to claim 1, wherein the digital signal processing means is a circuit board which is integrated into a computing system.

3. The system for detecting optical characteristics of biological tissue according to claim 1, wherein the photonic energy source is selected from the group comprising a laser, a laser diode, a light emitting diode, an arc flashlamp or a continuous wave bulb.

4. The system for detecting optical characteristics of biological tissue according to claim 1, wherein optical emission processing means and the received light optical processing means include one or more optical devices selected from the group comprising condensers, focusing devices, lenses, fibre optics, apertures and monochromators.

5. The system for detecting optical characteristics of biological tissue according to claim 1, wherein the optical detector is selected from the group comprising a gallium-arsenide photodiode, a cadmium sulfide photodiode or a silicon avalanche diode.

6. Use of an optical system for generating a pattern of optical characteristics of biological tissue, said optical characterstics being reflectance and fluorescence characteristics of the illuminated biological tissue, said optical system comprising:
(a) a photonic energy source for emitting electromagnetic radiation;
(b) an optical emission processing means for receiving the electromagnetic radiation from the photonic energy source and isolating one or more illumination wavelengths of the electromagnetic radiation, said optical emission processing means encoding the one or more illumination wavelengths using one or more pseudo random codewords or linear FM thereby generating an encoded signal, the optical emission processing means transmitting the encoded signal to the biological tissue;
(c) an optics assembly providing a means for aligning emitter optics of the optical emission processing means with detector optics of a received light optical processing means;
(d) a received light optical processing means for collecting and isolating one or more wavelengths of received electromagnetic radiation from the biological tissue created in response to the encoded signal and transmitting the one or more wavelengths of received electromagnetic radiation to an optical detector,
(e) an optical detector for sensing and converting the one or more wavelengths of received electromagnetic radiation into an electrical signal; and
(f) digital signal processing means for correlating the electrical signal received from the optical detector with the encoded signal thereby identifying an optical response of the biological tissue to the one or more illumination wavelengths, said digital signal processing means controlling the functionality of the photonic energy source, the optical emission processing means and the received light optical processing means.

7. A method for detecting one or more optical responses of biological tissue and creating a pattern of the one or more optical responses, said method comprising the steps of:
(a) generating one or more illumination wavelengths of electromagnetic radiation;
(b) encoding said one or more illumination wavelengths using one or more pseudo random codewords or linear FM thereby generating an encoded signal;
(c) illuminating the biological tissue with the encoded signal, in order to generate encoded reflectance and fluorescence from the biological tissue in response thereto;
(d) collecting said encoded reflectance and fluorescence;
(e) correlating said encoded reflectance and fluorescence with the encoded signal thereby identifying one or more optical responses to the one or more illumination wavelengths;
(f) repeating steps a) through e) for a next one or more wavelengths of electromagnetic radiation;
(g) generating a pattern of the one or more optical responses, said pattern being a representation of a particular one or more optical responses matched with a particular one or more illumination wavelengths.

8. The method for detecting one or more optical responses of biological tissue according to claim 7, wherein the pattern is a contour map, and a position on the contour map is represented by an illumination wavelength and a detection wavelength and intensity of the collected reflectance and fluorescence is represented by contours.

9. The method for detecting one or more optical responses of biological issue according to claim 7, wherein the pattern is a comparative pattern between detected optical responses of two biological tissue samples, said comparative pattern identifying optical response differences between the two biological tissue samples.

10. The method for detecting one or more optical responses of biological tissue according to claim 7, wherein the pattern is a three dimensional representation of the collected reflectance and fluorescence.

11. The method for detecting one or more optical responses of biological tissue and creating a pattern of the one or more optical responses according to claim 7, wherein the optical characteristics of biomarkers within the biological tissue are determined.

12. The method for detecting one or more optical responses of biological tissue according to claim 7, further comprising the step of determining a statistical significance value related to each of the one or more optical responses, said statistical significance value representative of a ratio of signal-to-noise determined during detection.

13. The method for detecting one or more optical characteristics responses of biological tissue according to claim 7, wherein the pattern is a comparative pattern between detected optical responses of an identical location of the biological tissue sample detected at different points in time, said comparative pattern identifying optical response differences of the biological tissue sample over time.

14. The method for detecting one or more optical characteristics response of biological tissue according to claim 7, wherein the pattern is a comparative pattern between detected optical responses of two or more different locations of the biological tissue sample, said comparative pattern identifying optical response differences of the two or more different locations of the biological tissue sample.
